(19) United States
(12) Patent Application Publication
(10) Pub. No.: US 2015/0066132 A1
(43) Pub. Date: Mar. 5, 2015

(54) BALLOON CATHETER AND STENT DELIVERY SYSTEM

(71) Applicant: TERUMO KABUSHIKI KAISHA, Shibuya-ku (JP)

(72) Inventor: Shinya Sawada, Fujinomiya-shi (JP)

(21) Appl. No.: 14/478,283

(22) Filed: Sep. 5, 2014

Related U.S. Application Data
(63) Continuation of application No. PCT/JP2013/052845, filed on Feb. 7, 2013.

(30) Foreign Application Priority Data
Mar. 14, 2012 (JP) 2012-057448

(51) Int. Cl. A61M 25/10; A61M 25/00; A61F 2/958

(52) U.S. Cl. CPC A61M 25/10184; A61F 2/958; A61M 25/003; A61M 25/1027; A61M 2025/1084; A61M 2025/004; A61M 2025/0031
USPC 623/1.11; 604/99.01; 29/446

(57) ABSTRACT

A balloon catheter is disclosed, which includes an elongated shaft body, a dilatable balloon in which a distal end portion and a proximal end portion form a dilation chamber inside of the dilatable balloon by being away from each other and being individually bonded to an outer periphery of the shaft body, and a stretch regulation member that connects the outer periphery of the shaft body or an inner periphery of the balloon of the distal end portion of the balloon and the outer periphery of the shaft body or the inner periphery of the balloon of the proximal end portion of the balloon with each other inside the dilation chamber while regulating a stretch occurring between the distal end portion and the proximal end portion of the balloon during dilation of the balloon.

### CROSS REFERENCES TO RELATED APPLICATIONS

This application is a continuation of International Application No. PCT/JP2013/052845 filed on Feb. 7, 2013, and claims priority to Japanese Application No. 2012-057448 filed on Mar. 14, 2012, the entire content of both of which is incorporated herein by reference.

### TECHNICAL FIELD

The present disclosure relates to a balloon catheter and a stent delivery system, and more specifically, to a balloon catheter and a stent delivery system used for treating a stenosed part or an occluded part occurring inside a living body lumen.

### BACKGROUND DISCUSSION

A balloon catheter is a medical device used for widening a lesion when the lesion such as a stenosed part or an occluded part occurs inside a living body lumen such as a blood vessel, a bile duct, the trachea, the esophagus, and the urethra. For example, for the medical treatment of myocardial infarction, angina pectoris, and the like which are caused due to an occurrence of a stenosed part in a coronary artery, for example, a method of widening the stenosed part from the inside of the stenosed part by using a balloon catheter is adopted.

This type of a balloon catheter generally has a structure in which a balloon is arranged in an outer peripheral portion on a distal end side of an elongated shaft body. After the shaft body is inserted into the living body lumen until the balloon is positioned in a lesion, the balloon is dilated in a radial direction to widen the lesion by supplying a dilation fluid into the balloon through the inside of the shaft body.

JP-A-2008-86463 discloses a balloon catheter in which a balloon 3 is attached to the shaft body having a dual-tube structure including an outer tube 1 and an inner tube 2, and which is illustrated in FIG. 8 of the present disclosure.

As shown in FIG. 8, the inner tube 2 has a forward extension portion 2a extending forward beyond a distal end opening portion 1a of the outer tube 1. A distal end portion 3a of the balloon 3 can be bonded to an outer periphery of the forward extension portion 2a of the inner tube 2 and a proximal end (“rear end”) portion 3b can be bonded to an outer periphery of the outer tube 1, thereby forming a dilation chamber 4 inside the balloon 3. The balloon 3 can be dilated by supplying a dilation fluid F to the dilation chamber 4 via a balloon dilation lumen 5, which can be formed between the outer tube 1 and the inner tube 2.

A balloon having a size in accordance with the part to be applied, the lesion, or the like is used as a balloon 3. However, generally, the maximum dilation pressure, which is the upper limit value of a dilation pressure that can be safely used, is set in advance in a balloon. The outer diameter of the balloon 3 during dilation can be adjusted by changing an injection pressure of a dilation fluid F, which is supplied to a dilation chamber 4 within a range not exceeding the maximum dilation pressure. For example, when the injection pressure of the dilation fluid F is increased, a dilation outer diameter of the balloon 3 can be widened as indicated by the two-dot chain line in FIG. 8. In this manner, the balloon 3 can be dilated until the target outer diameter is acquired while confirming the injection pressure of the dilation fluid F, and thus, the lesion is widened.

However, when the dilation outer diameter of the balloon 3 is widened by increasing the injection pressure of the dilation fluid F, the balloon 3 is also stretched in an axial direction together with an inner tube 2 causing a stretch E occurring between a distal end portion 3a and a proximal end portion 3b of the balloon 3. Thus, there is a possibility that the balloon 3 may also impose a burden to normal living body lumens other than the lesion.

The present disclosure provides a balloon catheter and a stent delivery system in which a stretch occurring between a distal end portion and a proximal end portion of a balloon can be relatively suppressed during dilation of the balloon.

### SUMMARY

In accordance with an exemplary embodiment, a balloon catheter is disclosed, which includes an elongated shaft body, a dilatable balloon in which a distal end portion and a proximal end portion form a dilation chamber inside of the dilatable balloon by being away from each other and being individually bonded to an outer periphery of the shaft body, and a stretch regulation member that connects the outer periphery of the shaft body or an inner periphery of the balloon in the vicinity of the distal end portion of the balloon and the outer periphery of the shaft body or the inner periphery of the balloon in the vicinity of the proximal end portion of the balloon with each other inside the dilation chamber while regulating a stretch occurring between the distal end portion and the proximal end portion of the balloon during dilation of the balloon.

In accordance with an exemplary embodiment, the shaft body can include an outer tube which has a distal end opening portion and an inner tube which is inserted into the outer tube to form a balloon dilation lumen between itself and an inner peripheral surface of the outer tube and has a forward extension portion extending forward beyond the distal end opening portion of the outer tube. The distal end portion of the balloon preferably forms a distal end side bonding portion by being bonded to an outer periphery of the forward extension portion of the inner tube. The proximal end portion of the balloon preferably forms a proximal end side bonding portion by being bonded to an outer periphery of the outer tube. The dilation chamber preferably communicates with the balloon dilation lumen.

In accordance with an exemplary embodiment, the stretch regulation member can be formed with a plurality of filaments which respectively extend between the distal end side bonding portion and the proximal end side bonding portion and are arranged in a circumferential direction of the shaft body at equal intervals.

The filaments can be formed of metallic materials or stretch-resistant resin materials.

In accordance with an exemplary embodiment, the stretch regulation members can be configured to include three filaments.

In accordance with an exemplary embodiment, a stent delivery system according to the present disclosure includes the balloon catheter disclosed above, and a stent that is arranged in an outer periphery of a balloon of the balloon catheter and expands in accordance with dilation of the balloon.

In accordance with an exemplary embodiment of the present disclosure, a stretch regulation member can connect an outer periphery of a shaft body or an inner periphery of a balloon in the vicinity of a distal end portion of the balloon and the outer periphery of the shaft body or the inner periphery of the balloon in the vicinity of a proximal end portion of the balloon with each other, and thus, a stretch occurring between the distal end portion and the proximal end portion of the balloon during dilation of the balloon can be relatively suppressed.

In accordance with an exemplary embodiment, a balloon catheter is disclosed comprising: an elongated shaft body, the shaft body including an outer tube which has a distal end opening portion, and an inner tube which is inserted into the outer tube to form a balloon dilation lumen between the inner tube and an inner peripheral surface of the outer tube and has a forward extension portion extending forward beyond the distal end opening portion of the outer tube; a dilatable balloon in which a distal end portion and a proximal end portion form a dilation chamber inside of the dilatable balloon by being away from each other and being individually bonded to an outer periphery of the shaft body; and a plurality of filaments that connect the outer periphery of the shaft body or an inner periphery of the balloon of the distal end portion of the balloon and the outer periphery of the shaft body or an inner periphery of the balloon of the proximal end portion of the balloon with each other inside the dilation chamber while regulating a stretch occurring between the distal end portion and the proximal end portion of the balloon during dilation of the balloon.

In accordance with an exemplary embodiment, a method is disclosed of regulating a stretch occurring between a distal end portion and a proximal end portion of a dilatable balloon during dilation, comprising: placing a stretch regulation member that connects an outer periphery of an elongated shaft body or an inner periphery of the balloon of the distal end portion of the balloon and an outer periphery of the elongated shaft body or an inner periphery of the balloon of the proximal end portion of the balloon with each other inside a dilation chamber of the dilatable balloon.

### BRIEF DESCRIPTION OF DRAWINGS

FIG. 1 is a side view illustrating an overall structure of a balloon catheter according to an exemplary embodiment of the disclosure.

FIG. 2 is a cross-sectional view illustrating a structure of a balloon in the balloon catheter in accordance with the exemplary embodiment as shown in FIG. 1.

FIG. 3 is a cross-sectional view taken along line A-A of FIG. 2.

FIG. 4 is a perspective view illustrating a structure inside the balloon in the balloon catheter in accordance with the exemplary embodiment as shown in FIG. 1.

FIG. 5 is a cross-sectional view illustrating a structure of the balloon in a balloon catheter in accordance with an exemplary embodiment of the disclosure.

FIG. 6 is a cross-sectional view illustrating a structure of the balloon in a balloon catheter in accordance with an exemplary embodiment of the disclosure.

FIG. 7 is a side view illustrating an overall structure of an exemplary stent delivery system of the disclosure.

FIG. 8 is a cross-sectional view illustrating a structure of the balloon in the balloon catheter in the related art.

### DETAILED DESCRIPTION

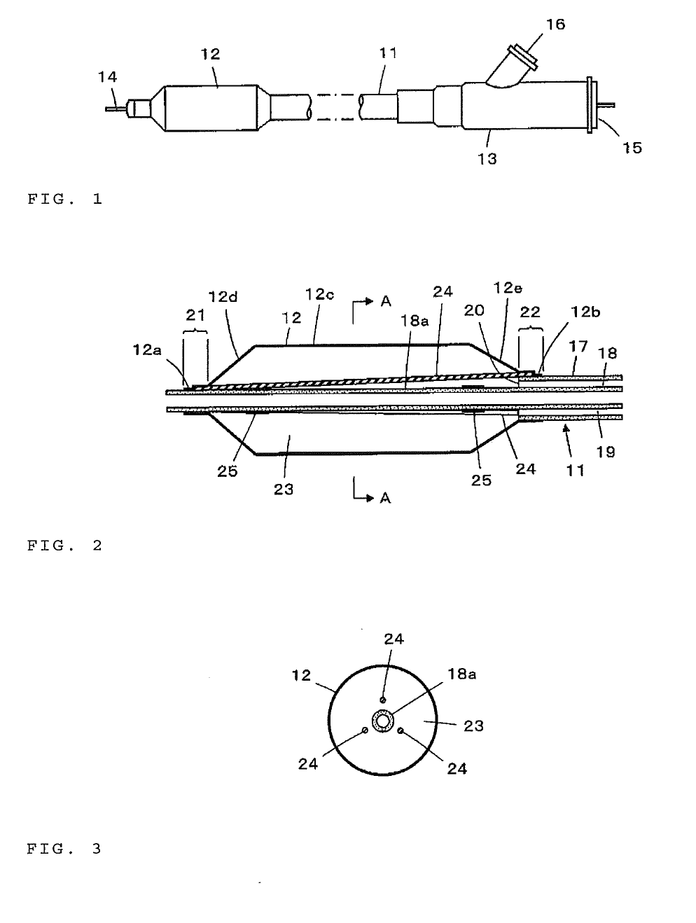

FIG. 1 illustrates an overall structure of a balloon catheter according to an exemplary embodiment. The balloon catheter can include an elongated shaft body 11. A dilatable balloon 12 is arranged to an outer peripheral portion of the distal end portion of the shaft body 11. A branch hub 13 is attached to a proximal end portion of the shaft body 11. The shaft body 11 and the balloon 12 are inserted into a living body lumen such as a blood vessel, a bile duct, the trachea, the esophagus and the urethra when treating a lesion. A guide wire 14 is inserted through the inside of the shaft body 11 to be able to move backward and forward. The branch hub 13 has an operation port 15 for making the guide wire 14 which is inserted through the inside of the shaft body 11 move backward and forward, and a fluid port 16 for injecting and suctioning a dilation fluid with respect to the balloon 12 via the inside of the shaft body 11.

As illustrated in FIG. 2, the shaft body 11 has a dual-tube structure including an outer tube 17 and an inner tube 18, which is inserted into the outer tube 17. A balloon dilation lumen 19 is formed between an outer peripheral surface of the inner tube 18 and an inner peripheral surface of the outer tube 17.

The balloon 12 has a middle portion 12c which is provided in the middle of the balloon 12 and of which an outer diameter is substantially uniform. The balloon 12 has a distal end portion 12a which is provided closer to a distal end side than the middle portion 12c and of which an outer diameter is smaller than that of the middle portion 12c, and a proximal end portion (“rear end portion”) 12b which is provided closer to a proximal or rear end side than the middle portion 12c and of which an outer diameter is smaller than that of the middle portion 12c. The balloon 12 also includes a distal end side tapered portion 12d which is provided between the distal end portion 12a and the middle portion 12c, and a proximal end side tapered portion (‘rear end side tapered portion”) 12e which is provided between the proximal end portion 12b and the middle portion 12c.

The outer tube 17 has a distal end opening portion 20, and the inner tube 18 has a forward extension portion 18a extending closer to the distal end side than the distal end opening portion 20 of the outer tube 17, that is, further forward of distal end opening portion 20 of the outer tube 17. A distal end side bonding portion 21 is formed by bonding the distal end portion 12a of the balloon 12 to an outer periphery of the forward extension portion 18a of the inner tube 18, and a proximal end side bonding portion (“rear end side bonding portion”) 22 is formed by bonding the proximal end portion 12b of the balloon 12 to an outer periphery of the distal end of the outer tube 17. Accordingly, a dilation chamber 23 communicating with the balloon dilation lumen 19 can be formed inside the balloon 12.

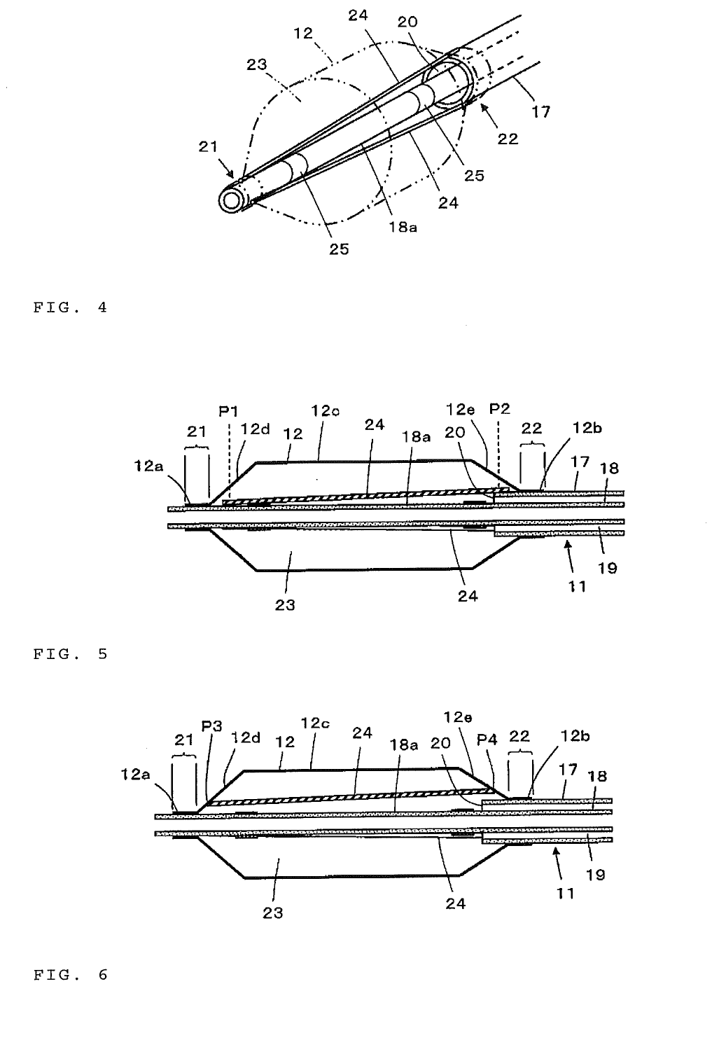

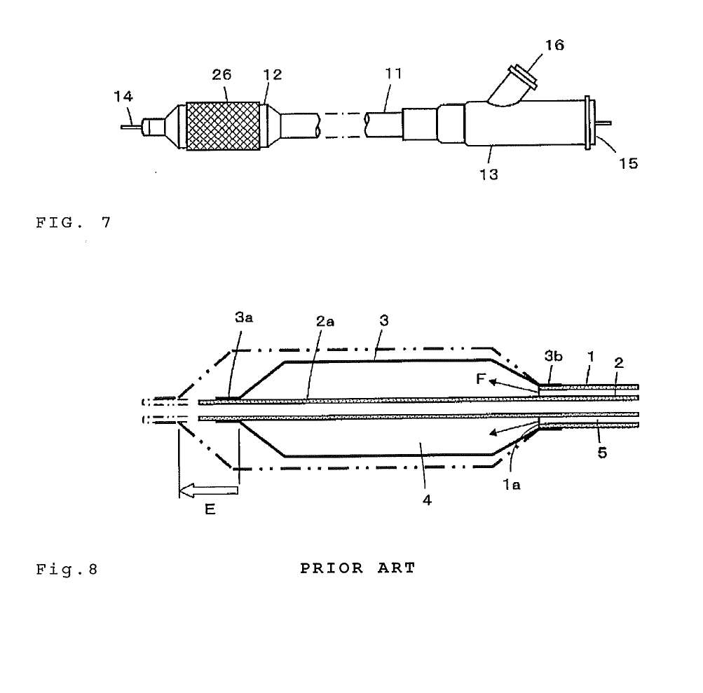

Inside the dilation chamber 23 of the balloon 12, the balloon 12 can include three filaments 24 extending between the distal end side bonding portion 21 and the proximal end side bonding portion 22. The three filaments 24 can be respectively fixed between the outer periphery of the inner tube 18 (forward extension portion 18a) and the inner periphery of the balloon 12 (distal end portion 12a) in the distal end side bonding portion 21, and between the outer periphery of the outer tube 17 and the inner periphery of the balloon 12 (proximal end portion 12b) in the proximal end side bonding portion 22. In accordance with an exemplary embodiment, the three filaments 24 connect the distal end side bonding portion 21 and the proximal end side bonding portion 22 with each other and function as stretch regulation members regulating a stretch occurring between the distal end portion 12a and the proximal end portion 12b of the balloon 12 during dilation of the balloon 12. In accordance with an exemplary embodiment, the filaments 24 can be arranged in a circumferential direction of the forward extension portion 18a of the inner tube 18 at equal intervals, as illustrated in FIG. 3.

Inside the dilation chamber 23 of the balloon 12 and on the outer periphery of the forward extension portion 18a of the inner tube 18, contrast markers 25 can be respectively arranged in the vicinity of the distal end portion 12a and the proximal end portion 12b of the balloon 12. In accordance with an exemplary embodiment, on account of these contrast markers 25, a position of the balloon 12 which is inserted into a living body lumen can be confirmed from the outside of a living body through an X-ray contrast.

The Inside of the inner tube 18 of the shaft body 11 communicates with the operation port 15 of the branch hub 13, and the guide wire 14 is inserted through the overall length of the inner tube 18 and the operation port 15.

The balloon dilation lumen 19 formed between the outer tube 17 and the inner tube 18 of the shaft body 11 communicates with the fluid port 16 of the branch hub 13 so that the dilation fluid can be injected to the inside of the dilation chamber 23 of the balloon 12 from the fluid port 16 via the balloon dilation lumen 19. In this case, the distal end side bonding portion 21 which is positioned on the outer periphery of the forward extension portion 18a of the inner tube 18 and the proximal end side bonding portion 22 which is positioned on the outer periphery in the vicinity of the distal end of the outer tube 17 are connected with each other by the filaments 24. As illustrated in FIG. 4, since a large gap can be formed between the filaments 24 adjacent to each other, the dilation fluid can be injected into the dilation chamber 23 of the balloon 12 from the balloon dilation lumen 19 through the distal end opening portion 20 of the outer tube 17 or can be suctioned from the dilation chamber 23 through the distal end opening portion 20 of the outer tube 17, without being disturbed by the filaments 24.

In accordance with an exemplary embodiment, for example, it can be preferable to use a material having certain flexibility as a material forming the outer tube 17 and the inner tube 18 of the shaft body 11. For example, the outer tube 17 and the inner tube 18 of the shaft body 11 can be a resin material such as polyolefin such as polyethylene, polypropylene, an ethylene-propylene copolymer and an ethylene-vinyl acetate copolymer, polyvinyl chloride, polyurethane, polyamide, a polyamide elastomer, polyimide, a silicon resin, polyether ether ketone, polyester, a polyester elastomer, and the like.

In accordance with an exemplary embodiment, for example, it can be preferable to form the balloon 12 with a high strength polymer, which can be stretchable. For example, the balloon 12 can be a resin material such as polyethylene terephthalate, polyester in which a main acid component or a main glycol component of polyethylene terephthalate is changed, a polyester elastomer, polyamide (nylon 12, nylon 11, MXD 6 nylon, and the like), a polyamide elastomer, polyarylene sulfide such as PPS (polyphenylene sulfide).

In accordance with an exemplary embodiment, for example, it can be preferable to form the filaments 24 used as the stretch regulation member with a material which has certain flexibility so as not to lessen softness in a portion corresponding to the balloon 12 while being unlikely to stretch against a tension force acting during dilation of the balloon 12. As a constituent material of the filament 24, for example, a metallic material or a stretch-resistant resin material can be used.

In accordance with an exemplary embodiment, since a metallic material generally has a greater Young's modulus than a resin material constituting the inner tube 18 and the balloon 12, the filament 24 is unlikely to stretch even though the filament 24 is formed to have a small diameter, thereby being suitable to be used as a constituent material of the filament 24. As a metallic material, for example, stainless steel such as SUS 304, SUS 316L can be used.

A stretch occurring between the distal end portion 12a and the proximal end portion 12b of the balloon 12 can be relatively suppressed when forming the filament 24 with a stretch-resistant resin material. In accordance with an exemplary embodiment, for example, it can also be effective when a fiber-reinforced resin, which is reinforced by a glass fiber, carbon fiber, and the like is used.

The contrast marker 25 can be formed of a material, which can be relatively easily, confirmed through an X-ray contrast, for example, gold, platinum, iridium, tungsten, an alloy of gold, platinum, iridium or tungsten, or a silver-palladium alloy.

When manufacturing a balloon catheter, the distal end portions of three filaments 24 can be in states of being respectively inserted between an inner peripheral surface of the distal end portion 12a of the balloon 12 and an outer peripheral surface of the forward extension portion 18a of the inner tube 18, and then, the inner peripheral surface of the distal end portion 12a of the balloon 12 can be fused on the outer peripheral surface of the forward extension portion 18a of the inner tube 18. Therefore, in accordance with an exemplary embodiment, the distal end side bonding portion 21 can be formed and each distal end portion of the filaments 24 can be fixed to the distal end side bonding portion 21 at the same time. Similarly, the proximal end portions (“rear end portions”) of three filaments 24 can be in states of being respectively inserted between an inner peripheral surface of the proximal end portion 12b of the balloon 12 and an outer peripheral surface in the vicinity of the distal end of the outer tube 17, and then, the inner peripheral surface of the proximal end portion 12b of the balloon 12 can be fused on the outer peripheral surface of the outer tube 17. Therefore, in accordance with an exemplary embodiment, the proximal end side bonding portion 22 can be formed and each proximal end portion of the filaments 24 can be fixed to the proximal end side bonding portion 22 at the same time.

In accordance an exemplary embodiment, operations of the exemplary embodiments as shown in FIGS. 1-4 are disclosed.

In accordance with an exemplary embodiment, initially, the guide wire 14 inserted into the inner tube 18 of the shaft body 11 from the operation port 15 is caused to protrude forward beyond the distal end portion of the inner tube 18, and the guide wire 14 serves as a leading guide for the balloon catheter to be inserted into the living body lumen such as a blood vessel. In this case, the dilation fluid is not yet injected into the dilation chamber 23 of the balloon 12 and the balloon 12 is in a folded state. An X-ray contrast is performed to recognize the contrast marker 25 arranged inside the dilation chamber 23 of the balloon 12 to confirm a position of the balloon 12 inside a living body lumen.

An insertion operation of the balloon catheter is completed, once the positioning of the balloon 12 is confirmed in the lesion requiring treatment. Here, the dilation fluid can be injected into the dilation chamber 23 of the balloon 12 through the balloon dilation lumen 19 from the fluid port 16. The balloon 12 can then be dilated until the target outer diameter is acquired while confirming the injection pressure of the dilation fluid. Accordingly, the lesion such as a stenosed part or an occluded part can be widened.

In this case, a tension force acts in the balloon 12 and the inner tube 18 along the axial direction in accordance with the injection pressure of the dilation fluid. However, since the distal end side bonding portion 21 which is positioned on the outer periphery of the forward extension portion 18a of the inner tube 18 and the proximal end side bonding portion 22 which is positioned on the outer periphery in the vicinity of the distal end of the outer tube 17 are connected with each other by three filaments 24, a stretch occurring between the distal end portion 12a and the proximal end portion 12b of the balloon 12 is regulated by the filaments 24. Accordingly, the balloon 12 can be relatively suppressed from being stretched in the axial direction.

For example, in this manner, it is possible to help prevent dilation of the balloon 12 in advance such that the balloon 12 does not impose a burden to normal living body lumens other than the lesion while dilating a lesion such as a stenosed part and an occluded part.

When the treatment of the lesion ends, the balloon 12 is folded by suctioning the dilation fluid from the inside of the dilation chamber 23 of the balloon 12 via the balloon dilation lumen 19, and then, in this state, the balloon catheter is drawn out from the inside of the living body lumen.

In accordance with an exemplary embodiment, the number of filaments 24 used as the stretch regulation member is not limited to three. For example, as long as the filament can regulate a stretch without lessening the softness of the balloon catheter in a portion corresponding to the balloon 12, it is possible to adopt equal to or less than two, or equal to or more than four filaments 24.

In accordance with an exemplary embodiment, in place of the filament 24, a member having a rod shape or a plate shape may be used as the stretch regulation member. However, in this case, it can be desirable not to lessen the softness of the balloon catheter in a portion corresponding to the balloon 12.

In the exemplary embodiment disclosed above, although both the end portions of each filament 24 are respectively fixed to the distal end side bonding portion 21 and the proximal end side bonding portion 22 of the balloon 12, it is not limited thereto. For example, as illustrated in FIG. 5, the distal end portion of each filament 24 can be fixed to be closer to a proximal end side than the distal end portion 12a (distal end side bonding portion 21) of the balloon 12, and specifically fixed to the outer periphery of the forward extension portion 18a of the inner tube 18 in a position P1 in the distal end side tapered portion 12d. The proximal end portion of each filament 24 can be fixed to be closer to the distal end side than the proximal end portion 12b (proximal end side bonding portion 22) of the balloon 12, and specifically fixed to the outer periphery of the outer tube 17 in a position P2 in the proximal end side tapered portion 12e.

In accordance with an exemplary embodiment, in this manner, even if the positions P1 and P2 of both the end portions of each filament 24 are slightly deviated from the distal end portion 12a and the proximal end portion 12b of the balloon 12, when the outer periphery of the forward extension portion 18a of the inner tube 18 in the vicinity (specifically, distal end side tapered portion 12d) of the distal end portion 12a of the balloon 12 and the outer periphery of the outer tube 17 in the vicinity (specifically, proximal end side tapered portion 12e) of the proximal end portion 12b of the balloon 12 are connected by each of the filaments 24, a stretch occurring between the position P1 and the position P2 can be relatively suppressed on account of an existence of the filaments 24 during dilation of the balloon 12, and thus, a certain degree of the stretch occurring between the distal end portion 12a and the proximal end portion 12b of the balloon 12 can be regulated.

In accordance with an exemplary embodiment, as shown in FIG. 5, the distal end portion of each of the filaments 24 may be fixed to the distal end side bonding portion 21, and the proximal end portion of each of the filaments 24 may be fixed to the outer periphery of the outer tube 17 in the position P2, or the distal end portion of each of the filaments 24 may be fixed on the outer periphery of the forward extension portion 18a of the inner tube 18 in the position P1, and the proximal end portion of each of the filaments 24 may be fixed to the proximal end side bonding portion 22.

As illustrated in FIG. 6, the distal end portion of each of the filaments 24 can be fixed to the inner periphery of the balloon 12 closer to the proximal end side than the distal end portion 12a (distal end side bonding portion 21) of the balloon 12, and specifically fixed to the inner periphery (position P3) of the distal end side tapered portion 12d, and the proximal end portion of each of the filaments 24 can be fixed to the inner periphery of the balloon closer to the distal end side than the proximal end portion 12b (proximal end side bonding portion 22) of the balloon 12, and specifically fixed to the inner periphery (position P4) of the proximal end side tapered portion 12e.

In this manner, for example, even though the positions P3 and P4 of both the end portions of each of the filaments 24 are slightly deviated from the distal end portion 12a and the proximal end portion 12b of the balloon 12, when the inner periphery of the balloon 12 in the vicinity (specifically, distal end side tapered portion 12d) of the distal end portion 12a of the balloon 12 and the inner periphery of the balloon 12 in the vicinity (specifically, proximal end side tapered portion 12e) of the proximal end portion 12b of the balloon 12 are connected by each of the filaments 24, a stretch occurring between the position P3 and the position P4 can be relatively suppressed on account of an existence of the filaments 24 during dilation of the balloon 12, and thus, a certain degree of the stretch occurring between the distal end portion 12a and the proximal end portion 12b of the balloon 12 can be regulated.

In accordance with an exemplary embodiment, as shown in FIG. 6, the distal end portion of each of the filaments 24 may be fixed to the distal end side bonding portion 21, the proximal end portion of each of the filaments 24 may be fixed to the inner periphery of the balloon 12 in the position P4, or the distal end portion of each of the filaments 24 may be fixed on the inner periphery of the balloon 12 in the position P3, and the proximal end portion of each of the filaments 24 may be fixed to the proximal end side bonding portion 22.

In the exemplary embodiment as disclosed, the description is given regarding a so-called over-the-wire-type balloon catheter in which the guide wire 14 is inserted through from the proximal end portion having the branch hub 13 attached thereto to the distal end portion, the disclosure can be similarly applied to a so-called rapid-exchange-type balloon catheter in which the guide wire is inserted through only the distal end portion.

As illustrated in FIG. 7, a stent 26, which expands in accordance with dilation of the balloon 12, may be arranged on the outer periphery of the balloon 12 of the balloon catheter according to the exemplary embodiments disclosed above in a stent delivery system.

The detailed description above describes a balloon catheter and a stent delivery system. The disclosure is not limited, however, to the precise embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A balloon catheter comprising:
an elongated shaft body;
a dilatable balloon in which a distal end portion and a proximal end portion form a dilation chamber inside of the dilatable balloon by being away from each other and being individually bonded to an outer periphery of the shaft body; and
a stretch regulation member that connects the outer periphery of the shaft body or an inner periphery of the balloon of the distal end portion of the balloon and the outer periphery of the shaft body or an inner periphery of the balloon of the proximal end portion of the balloon with each other inside the dilation chamber while regulating a stretch occurring between the distal end portion and the proximal end portion of the balloon during dilation of the balloon.

2. The balloon catheter according to claim 1,
wherein the shaft body includes:
an outer tube which has a distal end opening portion; and
an inner tube which is inserted into the outer tube to form a balloon dilation lumen between the inner tube and an inner peripheral surface of the outer tube and has a forward extension portion extending forward beyond the distal end opening portion of the outer tube;
wherein the distal end portion of the balloon forms a distal end side bonding portion by being bonded to an outer periphery of the forward extension portion of the inner tube;
wherein the proximal end portion of the balloon forms a proximal end side bonding portion by being bonded to an outer periphery of the outer tube; and
wherein the dilation chamber communicates with the balloon dilation lumen.

3. The balloon catheter according to claim 2,
wherein the stretch regulation member is formed with a plurality of filaments which respectively extend between the distal end side bonding portion and the proximal end side bonding portion and are arranged in a circumferential direction of the shaft body at equal intervals.

4. The balloon catheter according to claim 1,
wherein the stretch regulation member is formed with a plurality of filaments which respectively extend between the outer periphery of the shaft body of the distal end portion of the balloon and the outer periphery of the shaft body of the proximal end portion of the balloon and are arranged in a circumferential direction of the shaft body at equal intervals.

5. The balloon catheter according to claim 1,
wherein the stretch regulation member is formed with a plurality of filaments which respectively extend between an inner periphery of the balloon of the distal end portion of the balloon and the inner periphery of the balloon of the proximal end portion of the balloon and are arranged in a circumferential direction of the shaft body at equal intervals.

6. The balloon catheter according to claim 3,
wherein the filaments are formed of metallic materials or stretch-resistant resin materials.

7. The balloon catheter according to claim 3,
wherein the stretch regulation members comprises three filaments.

8. A stent delivery system comprising:
the balloon catheter according to claim 1; and
a stent that is arranged in an outer periphery of a balloon of the balloon catheter and expands in accordance with dilation of the balloon.

9. A balloon catheter comprising:
an elongated shaft body, the shaft body including an outer tube which has a distal end opening portion, and an inner tube which is inserted into the outer tube to form a balloon dilation lumen between the inner tube and an inner peripheral surface of the outer tube and has a forward extension portion extending forward beyond the distal end opening portion of the outer tube;
a dilatable balloon in which a distal end portion and a proximal end portion form a dilation chamber inside of the dilatable balloon by being away from each other and being individually bonded to an outer periphery of the shaft body; and
a plurality of filaments that connect the outer periphery of the shaft body or an inner periphery of the balloon of the distal end portion of the balloon and the outer periphery of the shaft body or an inner periphery of the balloon of the proximal end portion of the balloon with each other inside the dilation chamber while regulating a stretch occurring between the distal end portion and the proximal end portion of the balloon during dilation of the balloon.

10. The balloon catheter according to claim 9,
wherein the distal end portion of the balloon forms a distal end side bonding portion by being bonded to an outer periphery of the forward extension portion of the inner tube,
wherein the proximal end portion of the balloon forms a proximal end side bonding portion by being bonded to an outer periphery of the outer tube, and
wherein the dilation chamber communicates with the balloon dilation lumen.

11. The balloon catheter according to claim 9,
wherein the plurality of filaments are arranged in a circumferential direction of the shaft body at equal intervals.

12. The balloon catheter according to claim 9,
wherein a distal end portion of each of the plurality of filaments is fixed on a proximal end side of the distal end portion of the balloon; and
wherein a proximal end portion of each of the plurality of filaments is fixed on a distal end side of the proximal end portion of the balloon.

13. The balloon catheter according to claim 12,
wherein the distal portion of each of the plurality of filaments is fixed to the outer periphery of the forward extension portion of the inner tube in a distal end side tapered portion of the balloon;
wherein the proximal end portion of each of the plurality of filaments is fixed to the outer periphery of the outer tube in a proximal end side tapered portion of the balloon.

14. The balloon catheter according to claim 9,
wherein the balloon has a distal end side tapered portion and a proximal end side tapered portion;
wherein a distal end portion of each of the plurality of filaments is fixed to an inner periphery of the distal end side tapered portion of the balloon; and
wherein a proximal end portion of each of the plurality of filaments is fixed to the inner periphery of the proximal end side tapered portion of the balloon.

15. The balloon catheter according to claim 9,
wherein the plurality of filaments are formed of metallic materials or stretch-resistant resin materials.

16. The balloon catheter according to claim 9,
wherein the plurality of filaments comprises three filaments.

17. The balloon catheter according to claim 9 in combination with a stent that is arranged in an outer periphery of a balloon of the balloon catheter and expands in accordance with dilation of the balloon.

18. A method of regulating a stretch occurring between a distal end portion and a proximal end portion of a dilatable balloon during dilation, comprising:
placing a stretch regulation member that connects an outer periphery of an elongated shaft body or an inner periphery of the balloon of the distal end portion of the balloon and an outer periphery of the elongated shaft body or an inner periphery of the balloon of the proximal end portion of the balloon with each other inside a dilation chamber of the dilatable balloon.

19. The method of claim 18,
wherein the stretch regulation member is a plurality of filaments; and
arranging the plurality of filaments to extend between a distal end side bonding portion and a proximal end side bonding portion of the balloon in a circumferential direction of the shaft body at equal intervals.

20. The method of claim 19,
wherein the plurality of filaments comprises three filaments formed of metallic materials or stretch-resistant resin materials.
